(19) United States
(12) Patent Application Publication
(10) Pub. No.: US 2003/0208212 A1
(43) Pub. Date: Nov. 6, 2003

(54) Removable gastric band

(76) Inventor: Valerio Cigaina, Villorba (IT)

Correspondence Address: FITCH EVEN TABIN AND FLANNERY, 120 SOUTH LA SALLE STREET, SUITE 1600, CHICAGO, IL, 60603-3406, US

(21) Appl. No.: 10/148,421

(22) PCT Filed: Dec. 7, 2000
(86) PCT No.: PCT/US00/33236

(30) Foreign Application Priority Data
Dec. 7, 1999 (IT) M199A002641

(51) Int. Cl. A61B 17/08

(52) U.S. Cl. 606/151

(57) ABSTRACT

A removable gastric band is provided which can be used to control obesity by allowing control and/or modification of the diameter of a patient's stomach. More specifically, the present removable gastric band comprises an elongated body having a first or distal zone, a second or middle zone, a third or proximal zone and a closure mechanism, wherein the closure mechanism allows the elongated body to close around a portion of the stomach, preferably the proximal tract of the stomach, wherein the closure mechanism comprises at least one aperture in the first zone and a button in the second zone, and where the button can be inserted into the aperture to close the elongated body around, and hold it to, the portion of the stomach. The removable gastric band can be easily paired with the use of a gastric electrostimulator and may be useful, therefore, for inducing forced slimming in the initial phase of treatment for morbigenous obesity. Such electrostimulation devices may either be incorporated into the removable gastric band or located at a distance from the removable gastric band.

### RELATED APPLICATION

This application claims priority from Italian Patent Application Number MI99A002641, filed Dec. 7, 1999.

### FIELD OF THE INVENTION

The present invention relates to a removable gastric band which can be used to control obesity by allowing control and/or modification of the diameter of a patient's stomach.

### BACKGROUND OF THE INVENTION

Laparoscopic banding systems are available which provide for the use of an elongated main part that is placed around the stomach and closed over the stomach so as to reduce the diameter of the stomach to be able to treat the patient's obesity. Such currently available bands, however, present some drawbacks essentially due to the difficulty of application and/or removal of the gastric band. In fact, normally, the current bands' means of closing the elongated main part are almost always hard to manipulate; moreover, their connection entails the use of additional instruments and/or devices that further complicate the application and/or later removal of the gastric band for the surgeon.

Furthermore, to be able to remove the known bands, which must necessarily be done after a more or less long time interval, it is necessary to execute an additional surgical intervention and, consequently, to administer more anesthesia to the patient. The application and/or removal of the known bands also require the application of suture stitches, in addition to another intervention and more anesthesia. In particular, the bands used today are also hard to remove because they present little resistance to tissue adhesions and lack sufficient mechanical integrity to withstand tensile forces, both of which hinder their removal unless the patient is undergoing surgery.

It is desirable, therefore, to provide an improved gastric band which is both easier to implant within the patient and, when necessary, to remove from the patient.

### SUMMARY OF THE INVENTION

The present invention provides a removable gastric band which can be used to control obesity by allowing control and/or modification of the diameter of a patient's stomach. More specifically, the present invention provides a removable gastric band comprising an elongated body having a first or distal zone, a second or middle zone, a third or proximal zone and a closure mechanism, wherein the closure mechanism allows the elongated body to close around a portion of the stomach, preferably the proximal tract of the stomach, wherein the closure mechanism comprises a button in the first zone and at least one aperture in the second zone, such that the button can be inserted into the aperture to close the elongated body around, and hold it to, the portion of the stomach.

The present invention provides a removable gastric band comprising an elongated body having a first zone, a second zone, a third zone, and a closure mechanism, wherein the closure mechanism allows a portion of the elongated body to close around a section of the stomach, wherein the closure mechanism comprises a button in the first zone and at least one aperture in the second zone, such that the button can be inserted into the aperture to close the portion of the elongated body around, and hold it to, the section of the stomach, and wherein the portion of the elongated body is essentially planar in cross section.

The present invention also provides a method for treatment of obesity or for reducing weight in a patient, said method comprising:

(1) positioning a removable gastric band around a section of the patient's stomach;

(2) locking the removable gastric band around the section of the patient's stomach; and

(3) adjusting the removable gastric band to control the stomach's diameter in the section of the patient's stomach, wherein the removable gastric band comprises an elongated body having a first zone, a second zone, a third zone, and a closure mechanism,

wherein the closure mechanism allows a portion of the elongated body to close around the section of the patient's stomach, wherein the closure mechanism comprises a button in the first zone and at least one aperture in the second zone, such that the button can be inserted into the aperture to close the portion of the elongated body around, and hold it to, the section of the stomach, and wherein the portion of the elongated body is essentially planar in cross section.

The task proposed by the present invention is the realization of a removable gastric band that eliminates the above-noted drawbacks of the known gastric bands. Within the scope of this task, one important purpose of the invention is to realize a removable gastric band that can be removed without having to subject the patient to further intervention and, consequently, to additional general anesthesia.

Yet another purpose of the invention is to realize a removable gastric band that is easy to remove because it is highly resistant to adhesion to the tissue and has sufficient mechanical integrity to withstand tensile forces during removal. Yet another purpose of the invention is to realize a removable gastric band that can be applied without necessarily having to use suture stitches.

Yet another purpose of the invention is to realize a removable gastric band that can be applied and/or removed by the surgeon very simply and without having to use additional instruments or devices for that purpose. Another purpose of the invention is to realize a removable gastric band that is extremely easy to manipulate, so that it can be easily placed in and/or removed from the patient.

### BRIEF DESCRIPTION OF THE DRAWINGS

FIG. 1 illustrates the gastric band according to the invention.

FIG. 2 shows schematically the gastric band according to the invention being applied to the proximal tract of a patient's stomach;

FIG. 3 shows the gastric band according to the invention applied to the proximal tract of the patient's stomach;

FIG. 4 shows the gastric band according to the invention inflated so as to compress a portion of the patient's stomach;

FIG. 5A shows in cross-sectional view (along line A-A in FIG. 1) the inner surface of the gastric band compressing the patient's stomach before the gastric band has been inflated;

FIG. 5B shows in cross-sectional view (along line A-A in FIG. 1) the inner surface of the gastric band in relation to the patient's stomach after the gastric band has been inflated;

FIG. 5C shows in cross-section sectional view a reinforcing member or element located within the elongated perimeter (i.e., the rib connecting the inner and outer surfaces, thereby forming an inflatable chamber or cavity) of the gastric band which reduces the tendency of the gastric band to twist around its longitudinal axis;

FIG. 6A is a view of the gastric band attached to the patient's stomach with the inflation mechanism positioned to allow for inflation; and

FIG. 6B is an expanded view of the inflation mechanism.

### DETAILED DESCRIPTION OF THE INVENTION

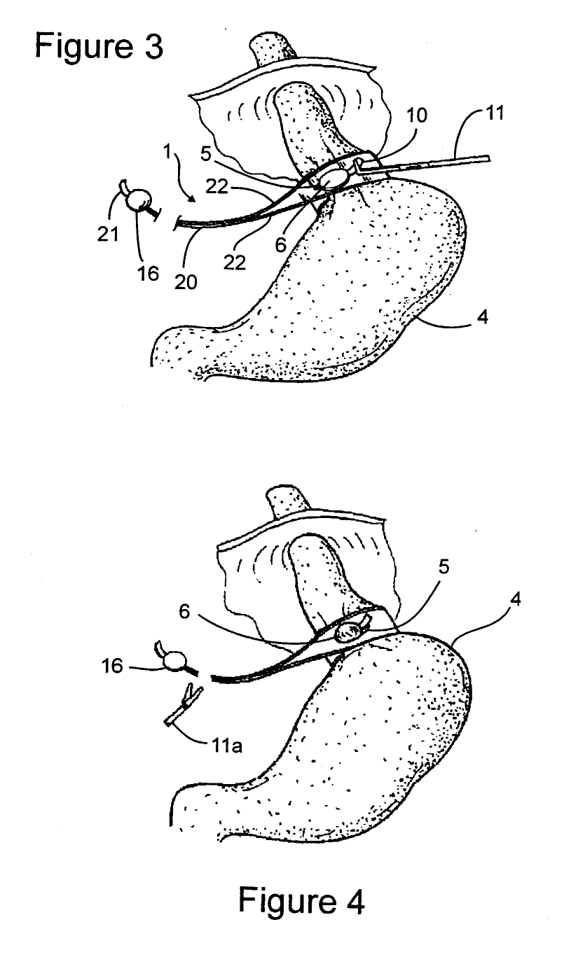

With reference to the figures described above, the removable gastric band according to the invention, indicated as a whole with reference number 1, comprises an elongated body 3 having a first or distal zone 30, a second or middle zone 32, a third or proximal zone 34, and a closure mechanism 2 for closing the elongated body 3 back upon itself so as to surround a portion, preferably the proximal tract, of the patient's stomach 4. The closure mechanism 2 preferably comprises a button 6 in the first zone 30 and a corresponding aperture 5 in the second zone 32 whereby the button 6 can fit through the aperture 5 and fix or lock the elongated body 3 back onto to itself. Once locked into place, the gastric band 1 completely encircles and compresses a portion of the patient's stomach (see, e.g., FIGS. 3 and 4). Although only one aperture 5 is shown within the second zone 32, a plurality of such apertures can be provided if desired; using such a plurality of apertures allows the surgeon to more closely adjust the diameter of the encircling portion of the gastric band to the particular patient's situation and needs.

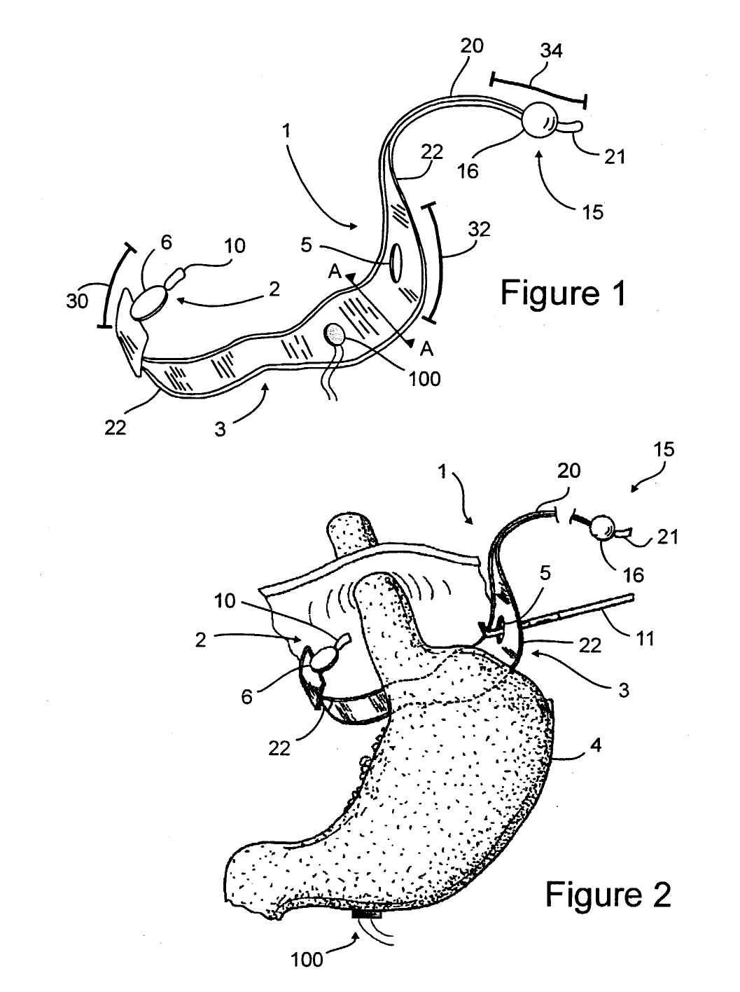

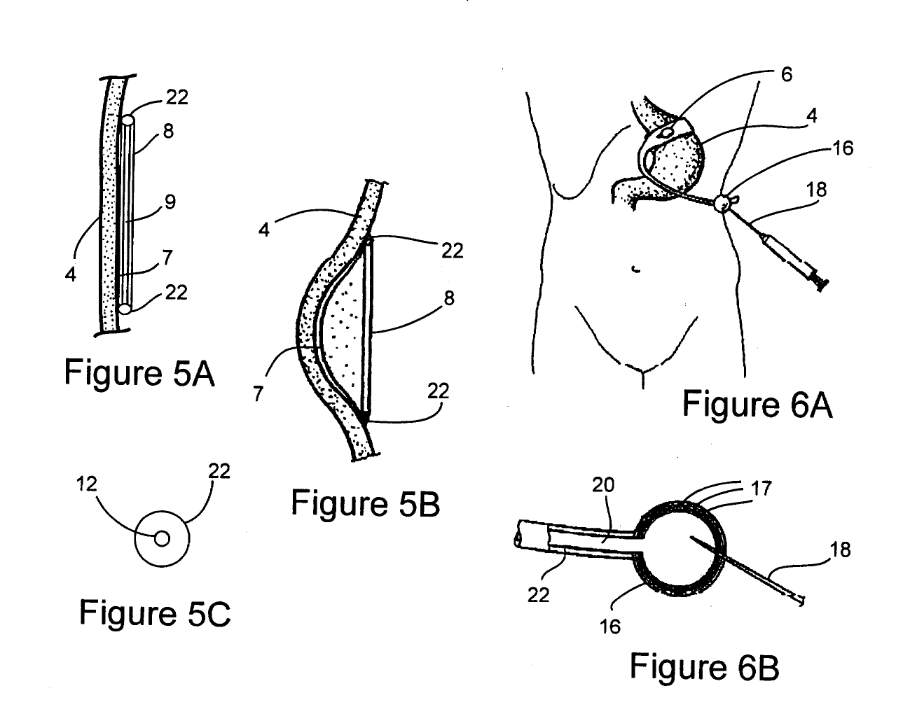

Appropriately, button 6 is suitably shaped and sized to allow it to be internally introduced into aperture 5, as well as to close, in an extremely simple but secure manner, the elongated main part 3 around stomach 4 and keep it in place. Although the button 6 and aperture 5 are preferably circular as shown in FIG. 1, other shapes can be used so long as they provide the desired closing/locking action. Elongated body 3 presents at least an inner surface 7 and an outer surface 8 as more clearly shown in FIG. 5A (deflated state) and FIG. 5B (inflated state). Preferably, the elongated body 3 has an inflatable portion or internal cavity 9 formed by inner surface 7, outer surface 8, and ribs, welds, or closures 22 at the edges of the elongated body 3. Ribs 22 essentially form a closed space or internal cavity 9 in combination with the inner and outer surfaces 7 and 8 (see, e.g., FIG. 5B). Such an inflatable member allows the elongated body 3 to be expanded when a physiological inflation medium (i.e., liquid or gas) 9 is introduced between inner surface 7 and outer surface 8. (Both the inflatable portion or internal cavity and the inflation medium, which effectively defines the size of the internal cavity, are referred to by common reference number 9 in the figures.) Preferably, button 6 is fluid-dynamically connected to inner surface 7 of elongated body 3 in such a way that, as the latter inflates, button 6 also inflates, as can be seen, for example, in FIG. 3; this provides a more secure locking of the elongated body back upon itself. Preferably, the inner surface 7 is more easily expandable relative to outer surface 8 so that inflation of the elongated body 3 allows further compression, and thus more control of the compression, of the stomach. Generally, therefore, it is preferred that outer surface 8 undergoes little, if any, expansion when the physiological inflation liquid or gas 9 is introduced between inner surface 7 and outer surface 8. Compression of the stomach using the gastric band of the present invention allows for a reduction of the stomach volume as desired. The degree of compression can be modified as desired throughout the course of treatment by adding or removing inflation medium 9.

Furthermore, button 6 and aperture 5 are preferably sized relative to one another that once button 6 is passed through aperture 5 and inflated, the closure mechanism is securely activated but, once button 6 is deflated, the closure mechanism can easily be deactivated by simply pulling on one end of the gastric band (preferably by pulling on tube 20) to remove the gastric band from the abdomen. Furthermore, button 6 is preferably located outside of elongated main part 3 by a distance that can allow a substantial alignment of the first and second zones of elongated body 3, when the latter is closed around the stomach. Thus, when the elongated body 3 is inflated (and preferably button 6 is also inflated), there is no unsuitable and/or harmful superposition of two parts of the elongated body 3 that would provide an undesired enlargement at the zone where they are superposed. In other words, the inflatable portions of the gastric band do not overlap; such overlapping might result in undesirable and/or additional stomach compression in the area of overlap.

Preferably, button 6 is equipped with flap 10 that makes it easier to catch and insert the button 6 into aperture 5 using appropriate instruments. Flap 10 is appropriately made with no internal cavity and, therefore, is not inflatable. Flap 10 can be grasped quickly and simply by surgical endoscopic forceps 11 that is passed first through aperture 5 (see FIG. 2). Once grasped, flap 10 and button 6 are pulled back through aperture 5 to lock the gastric band in place (see FIG. 3).

As noted above, it is preferred that the button 6 expands at the same time as inner surface 7 of the elongated body 3. The expansion of button 6 should, however, be limited so that, once the gastric band 1 is locked firmly in place, the button 6 does not under go significant further expansion. For example, the relative thicknesses of the walls of the button 6 and inner surface 7 can be controlled such that the inflation of the button will reach a definite value without expanding any further, independently of the inflation of inner surface 7 of elongated body 3. Thus, preferably the button 6 expands to a size sufficient to lock the closure mechanism 2 in place but not significantly larger.

The elongated body 3 is preferably designed so as to prevent or reduce the tendency of the elongated body 3 to rotate around its long axis as it is being placed in the proper position around the patient's stomach. For example, one or both of the ribs 22 at the edges of the elongated body 3 can contain stiffening elements 12 (see FIG. 5C) which will reduce the tendency of the elongated body 3 to rotate or twist about its long axis without effecting the ability of the elongated body to fold back on itself and encircle the patient's stomach. Such stiffening elements 12 will reduce the tendency to twist as the gastric band is being positioned within the patient. Such stiffening or antirotation elements 12 will tend to stabilize the prosthesis and make the insertion easier. The ribs 22 at the edges of the elongated body 3 are preferably gently curved so as not to create problems either at the time of the implant or during removal by pulling of elongated body 3 from the outside; in other words, the ribs, as well as other portions of the gastric band, preferably present smooth and gently curved surfaces to allow the gastric band to slide easily around organs during implantation and removal.

The gastric band preferably has an inflation mechanism 15 comprising a reservoir 16 for receiving the inflation medium, preferably a physiological liquid or gas, for inflating both elongated body 3 and button 6. Preferably, the reservoir 16 has several concentric layers 17 to allow it to be pierced, for example with needle 18, without the inflation medium 9 being able to escape from the perforation. Preferably, reservoir 16 is constructed with multiple layers of material (preferably elastomeric or plastic materials) that, when pricked with needle 18, allows the hole to be made without skewing or leakage between the different layers 17. Such skewing or leakage would generally be mainly noticeable or chiefly accentuated during the expansion of reservoir 16 when the inflation medium 9 would tend to leak. The external layer of reservoir 16, preferably constructed of biocompatible materials, is generally thicker than the other, internal layers and can even be rigid, since it preferably remains adjacent to the abdominal wall, more preferably within the subcutis, and presents such dimensions as to permit easy introduction through a surgical laparoscopic trocar. By maintaining the reservoir 16 near the abdominal wall, the compression of the stomach can more easily be modified as desired by addition or removal of the inflation medium 9. In some instances, it may be desired for the reservoir 16 to remain outside the abdominal wall.

The elongated body 3 can be inflated using the inflation medium introduced into the reservoir 16 using, for example, a syringe 18 as shown in FIGS. 6A and 6B. The elongated body is inflated until the desired degree of compression of the stomach occurs. The inflation of the gastric band is generally performed under the control of the endoscopist, who can observe, preferably using an endoscope from inside the stomach, the diameter of the gastric restriction induced by the inflation of the gastric band, particularly by inner surface 7. Preferably, essentially the entire length of the gastric band 3 encircling the stomach can be inflated using the inflation medium 9.

Reservoir 16 is preferably located in the third or proximal zone 34 of elongated body 3 and is connected to the second or middle zone 32 containing aperture 5 is present via tube 20. The length of tube 20 can be varied as needed for particular patients; preferably, tube 20 does not significantly expand when inflation medium 9 is added to the gastric band. In operation, the reservoir 16 is preferably not secured and remains in the subcutis of the abdominal wall. It may be located, using, for example, feel or ultrasound, for introduction of the inflation medium in order to inflate or deflate the gastric band. Using such a technique, the diameter of the gastric constriction provided by the gastric band can be modified or adjusted as desired. Preferably, reservoir 16 has a flap 21 which can be grasped using appropriate instruments to assist in the inflation or deflation operation.

Preferably, both the main portion of the elongated body 3 and the tube 20 have stiffening or antirotation elements 12 within the ribs 22 as shown in FIG. 5C. For example, the stiffening elements 12 could be a thin steel, other metal, or other type wire that is fused into the plastic material of the rib 22. Such a stiffening element 12 reduces the tendency of the gastric band to rotate about its long axis before the closure mechanism is activated. Additionally, it makes the gastric band considerably stronger (i.e., acting as a reinforcing element); this added strength may be especially important when the gastric band is removed from the patient by pulling on the proximal end 34 from the outside. The stiffening element 12, when formed using a steel or other suitable metal wire, can also be observed using X-rays, thereby determining the exact position of the band inside the patient's abdomen. Preferably, such stiffening element 12 extends essentially the entire length of the elongated body 3 (i.e., through the first, second, and third zones, including tube 20).

When it is desired to remove the gastric band from the abdomen, it is generally preferred to remove at least a portion of the inflation medium 9 so that the closure mechanism 2 can more easily be disengaged. A significant portion of the inflation medium 9 can be removed using, for example, a syringe using essentially the same procedures as used for the initial inflation process. Alternatively, tube 20 can be cut using cutting device 11a to separate reservoir 16, as represented in FIG. 3, to release inflation medium 9. Preferably, at least a portion of inflation medium 9 is removed prior to cutting tube 20 so as to minimize release of inflation medium 9 into the abdominal cavity. For this purpose, under local anesthesia, a small cutaneous incision is made in the abdominal wall to access reservoir 16, at which time tube 20 is cut and the reservoir 16 is removed from the abdominal cavity. After the closure mechanism 2 is disengaged, the gastric band 3 can be removed from the abdominal cavity by pulling on the tube 20 through the small cutaneous incision.

Preferably, the limit of expandability of inner surface 7 is linked to the limit of compressibility of the gastric walls and the two ends of the elongated body must be blunted enough to allow sliding between the patient's tissues in the phase of removal from the abdomen. In the removal phase, the gastric band will behave as an abdominal drainage tube. Preferably, the materials of construction and the surface smoothness are such that they will impede the production of fibrotic scar adhesions, as normally occurs with drainage tubes or prostheses of silicone materials. Such a smooth surface helps to prevent tissue adhesion to the gastric band. Thus, once deflated and unbuttoned, the gastric band can be removed easily by pulling on one end through a small incision. Preferably, the gastric band will have sufficient strength to withstand the forces associated with removal by this technique.

The gastric band of the present invention can be easily paired with the use of a gastric electrostimulator 100 and may be useful, therefore, for inducing forced slimming in the initial phase of treatment for morbigenous obesity. The electrostimulator 100 may be incorporated into the design of the gastric band as shown in FIG. 1 (i.e., attached to the inner surface 7) such that the electrostimulator 100 is in contact with the stomach when the gastric band is properly positioned. Alternatively, it may be separately implanted elsewhere within the abdominal cavity as shown in FIG. 2 (e.g., attached to the antrum). If incorporated into the gastric band design, the electrostimulator 100 is implanted at the same time as, and held in place by, the gastric band, thereby eliminating separate attachment of the electrostimulator 100. In such a unitary design, however, the electrostimulator 100 must be removed at the same time as the gastric band. If such an electrostimulator 100 is separately placed at a distance from the gastric band, it may remain within the abdominal cavity after removal of the gastric band. The selection of the preferred location of such an electrostimulator 100 relative to the gastric band will depend largely on the particular patient's requirements and planed treatment regime. Both the electrostimulator 100 and the gastric band are preferably installed and/or removed at the same time, thereby reducing the extent of surgical intervention and anesthesia.

Conventional electrostimulation devices 100 may be used in the practice of this invention in combination with the gastric band 3. Such devices include, for example, those described in U.S. Pat. No. 5,423,872 (Jun. 3, 1995) (an implantable gastric electrical stimulator at the antrum area of the stomach which generates sequential electrical pulses to stimulate the entire stomach, thereby artificially altering the natural gastric motility to prevent emptying or to slow down food transit through the stomach); U.S. Pat. No. 5,690,691 (Nov. 25, 1997) (a portable or implantable gastric pacemaker employing a number of electrodes along the greater curvature of the stomach for delivering phased electrical stimulation at different locations to accelerate or attenuate peristaltic movement in the gastrointestinal tract); U.S. Pat. No. 5,836,994 (Nov. 17, 1998) (an implantable gastric stimulator which incorporates direct sensing of the intrinsic gastric electrical activity by one or more sensors of predetermined frequency bandwidth for application or cessation of stimulation based on the amount of sensed activity); U.S. Pat. No. 5,861,014 (Jan. 19, 1999) (an implantable gastric stimulator for sensing abnormal electrical activity of the gastrointestinal tract so as to provide electrical stimulation for a preset time period or for the duration of the abnormal electrical activity to treat gastric rhythm abnormalities); U.S. Pat. No. 6,041,258 (Mar. 21, 2000) (electrostimulation device with improved handle for laparoscopic surgery); U.S. patent application Ser. No. 09/640,201 (filed Aug. 16, 2000) (eletrostimulation device attachable to enteric or endo-abdominal tissue or viscera which is resistance to detachment); PCT Application Serial Number PCT/US00/09910 (filed Apr. 14, 2000; Attorney Docket No. 3581/006 PCT) entitled “Gastric Stimulator Apparatus and Method for Installing” based on U.S. Provisional Application Serial Nos. 60/129,198 and 60/129,199 (both filed Apr. 14, 1999); PCT Application Serial Number PCT/US00/10154 (filed Apr. 14, 2000; Attorney Docket No. 3581/004 PCT) entitled “Gastric Stimulator Apparatus and Method for Use” based on U.S. Provisional Application Serial Nos. 60/129,209 (filed Apr. 14, 1999) and 60/466,387 (filed Dec. 17, 1999); and U.S. Provisional Patent Application Serial No. 60/235,660 (filed Sep. 26, 2000) entitled “Method and Apparatus for Intentional Impairment of Gastric Motility and/or Efficiency by Triggered Electrical Stimulation of the Gastric Tract with Respect to the Intrinsic Gastric Electrical Activity.” All of these patents, patent applications, provisional patent applications, and/or publications are hereby incorporated by reference.

Moreover, the gastric band of the invention is of great clinical interest, especially in relation to problems inherent to prolonged permanence in the abdomen, that is, intragastric decubitus, perforation, strangulation, and the like. In practice it has been confirmed that the removable gastric band according to the invention is particularly advantageous because it can be removed without having to perform an additional surgical intervention and additional anesthesia on the patient, thanks especially to its qualities of resistance to pulling.

The invention thus conceived is susceptible to numerous modifications and variations, all falling within the scope of the inventive concept; furthermore, all of the details can be substituted with technically equivalent elements. In practice, other materials and dimensions can be used, depending on the demands and on the state of the technique.

That which is claimed is:

1. A removable gastric band comprising an elongated body having a first zone, a second zone, a third zone, and a closure mechanism, wherein the closure mechanism allows a portion of the elongated body to close around a section of the stomach, wherein the closure mechanism comprises a button in the first zone and at least one aperture in the second zone, such that the button can be inserted into the aperture to close the portion of the elongated body around, and hold it to, the section of the stomach, and wherein the portion of the elongated body is essentially planar in cross section.

2. The removable gastric band of claim 1, wherein at least the portion of the elongated body encircling the section of the stomach comprises an essentially planar inner surface, an essentially planar outer surface, and ribs running along the elongated body and connecting the inner and outer surfaces to form an internal cavity, such that the cavity can be inflated whereby the inner surface can controllably compress the section of the stomach.

3. The removable gastric band of claim 2, wherein the button is fluid-dynamically connected to the cavity and is inflatable, whereby the elongated body can be more securely closed around the section of the stomach when the cavity is inflated.

4. The removable gastric band of claim 3, wherein the button is located outside of the elongated body by a distance to allow substantial alignment of the first and second zones of the elongated body when closed around said stomach.

5. The removable gastric band of claim 3, wherein the button has a flap for catching and easy introduction into the aperture.

6. The removable gastric band of claim 5, wherein the ribs have reinforcing elements to reduce the tendency of the elongated body to rotate around its long axis.

7. The removable gastric band of claim 6, wherein the second and third zones are connected by a tube and the third zone has a reservoir for receiving an inflation medium and wherein the reservoir is fluid-dynamically connected to the cavity, whereby the cavity can be inflated or deflated by adding or removing, respectively, inflation medium from the reservoir.

8. The removable gastric band of claim 7, wherein the reservoir comprises a sphere having a plurality of concentric layers to allow the reservoir to be pierced with a needle without allowing the inflation medium to escape.

9. The removable gastric band of claim 8, wherein the reinforcing elements are radiopaque.

10. The removable gastric band of claim 7, wherein the reservoir has a flap for easy holding.

11. The removable gastric band of claim 8, wherein the reservoir has a flap for easy holding.

12. The removable gastric band of claim 2, wherein essentially planar inner surface of the portion of the elongated body encircling the section of the stomach has an electrostimulator that contacts the stomach when the gastric band is in placed around the stomach.

13. The removable gastric band of claim 7, wherein the essentially planar inner surface of the portion of the elongated body encircling the section of the stomach has an electrostimulator that contacts the stomach when the gastric band is in place around the stomach.

14. A method for treatment of obesity in a patient, said method comprising:
(1) positioning a removable gastric band around a section of the patient's stomach;
(2) locking the removable gastric band around the section of the patient's stomach; and
(3) adjusting the removable gastric band to control the stomach's diameter in the section of the patient's stomach, wherein the removable gastric band comprises an elongated body having a first zone, a second zone, a third zone, and a closure mechanism,
wherein the closure mechanism allows a portion of the elongated body to close around the section of the patient's stomach, wherein the closure mechanism comprises a button in the first zone and at least one aperture in the second zone, such that the button can be inserted into the aperture to close the portion of the elongated body around, and hold it to, the section of the stomach, and wherein the portion of the elongated body is essentially planar in cross section.

15. The method of claim 14, wherein at least the portion of the elongated body encircling the section of the stomach comprises an essentially planar inner surface, an essentially planar outer surface, and ribs running along the elongated body and connecting the inner and outer surfaces to form an internal cavity, such that the cavity can be inflated whereby the inner surface can controllably compress the section of the stomach.

16. The method of claim 15, wherein the button is fluid-dynamically connected to the cavity and is inflatable, whereby the elongated body can be more securely closed around the section of the stomach when the cavity is inflated.

17. The removable gastric band of claim 16, wherein the button is located outside of the elongated body by a distance to allow substantial alignment of the first and second zones of the elongated body when closed around said stomach.

18. The method of claim 16, wherein the button has a flap for catching and easy introduction into the aperture.

19. The method of claim 18, wherein the ribs have reinforcing elements to reduce the tendency of the elongated body to rotate around its long axis.

20. The method of claim 19, wherein the second and third zones are connected by a tube and the third zone has a reservoir for receiving an inflation medium and wherein the reservoir is fluid-dynamically connected to the cavity, whereby the cavity can be inflated or deflated by adding or removing, respectively, inflation medium from the reservoir.

21. The method of claim 20, wherein the reservoir comprises a sphere having a plurality of concentric layers to allow the reservoir to be pierced with a needle without allowing the inflation medium to escape.

22. The method of claim 21, wherein the reinforcing elements are radiopaque.

23. The method of claim 20, wherein the reservoir has a flap for easy holding.

24. The method of claim 21, wherein the reservoir has a flap for easy holding.

25. The method of claim 15 further comprising implanting an electrostimulator near or adjacent to the patient's stomach and providing electrostimulation to the patient's stomach in combination with the gastric band.

26. The method of claim 25, wherein the electrostimulator is located on the essentially planar inner surface of the portion of the elongated body encircling the section of the patient's stomach such that the electrostimulator contacts the patient's stomach when the gastric band is in place around the patient's stomach.

27. The method of claim 25, wherein the electrostimulator is located separately from the gastric band.

28. The method of claim 20 further comprising implanting an electrostimulator near or adjacent to the patient's stomach and providing electrostimulation to the patient's stomach in combination with the gastric band.

29. The method of claim 28, wherein the electrostimulator is located on the essentially planar inner surface of the portion of the elongated body encircling the section of the patient's stomach such that the electrostimulator contacts the patient's stomach when the gastric band is in place around the patient's stomach.

30. The method of claim 28, wherein the electrostimulator is located separately from the gastric band.
